(19) United States
(12) Patent Application Publication
(10) Pub. No.: US 2025/0002850 A1
(43) Pub. Date: Jan. 2, 2025

(54) METHODS FOR MANUFACTURING T CELLS EXPRESSING OF CHIMERIC ANTIGEN RECEPTORS AND OTHER RECEPTORS

(71) Applicant: City of Hope, Duarte, CA (US)

(72) Inventors: Darya Alizadeh, Duarte, CA (US); Christine E. Brown, Duarte, CA (US); Stephen J. Forman, Duarte, CA (US)

(21) Appl. No.: 18/421,826

(22) Filed: Jan. 24, 2024

Related U.S. Application Data
(63) Continuation of application No. 16/465,847, filed on May 31, 2019, now abandoned.
(60) Provisional application No. 62/429,665, filed on Dec. 2, 2016.

(51) Int. Cl. C12N 5/0783; A61K 39/00

(52) U.S. Cl. CPC C12N 5/0636; A61K 39/4611; A61K 39/4631; A61K 39/464412; A61K 2239/48; C12N 2501/2302; C12N 2501/2307; C12N 2501/2315; C12N 2501/2321; C12N 2501/505; C12N 2501/515; C12N 2501/599

(57) ABSTRACT

Methods for preparing and expanding T cells comprising central memory T cells. memory stem T cells, and naïve T cells and expressing a chimeric antigen receptor by culturing in the presence of IL-15 and low or no exogenously added IL-2 and IL-7 arc described.

### CROSS-REFERENCE TO RELATED APPLICATION

This application is a continuation of U.S. application Ser. No. 16/465,847, filed May 31, 2019, which is a U.S. National Stage Application under 35 U.S.C. § 371 and claims benefit of International Application No. PCT/US2017/064326, filed Dec. 1, 2017, which claims the benefit of U.S. Application Ser. No. 62/429,665, filed Dec. 2, 2016. The disclosure of the prior applications are considered part of and are incorporated by reference in the disclosure of this application.

### STATEMENT REGARDING FEDERALLY SPONSORED RESEARCH

This invention is made with government support in the [symbol] The United States government has certain rights in the invention.

### BACKGROUND

Adoptive T cell therapy (ACT) utilizing ex vivo expanded autologous and allogeneic T cells is an attractive therapeutic approach for the treatment of viral infection, cancer and autoimmune disease. Methods that enable the rapid generation of large numbers of therapeutic T cells are critical to the potency and safety of ACT. Various T cell enrichment methods, including selection of defined T cell subsets, as well as expansion methods have been used for ACT. It is desirable to employ a T cell population that permits relatively high activity in vivo and relatively high proliferation potential.

### SUMMARY

Described herein is a method for manufacturing T cell populations useful in T cell therapy, for example, T cells expressing a recombinant T cell receptor (e.g., a chimeric antigen receptor (“CAR”) or T cell receptor (“TCR”)) or tumor infiltrating lymphocytes (“TIL”). The T cell populations are also useful for a variety of purposes requiring a highly active, long-lived T cell population. The methods described herein entail expanding T cell populations in the presence of exogenously added IL-15 and in presence of minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml). In some cases, the cells are expanded in the presence of exogenously added IL-15 (e.g., at least 10 ng/ml) and minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml) and minimal or no exogenously added IL-7 (e.g., less than 10 ng/ml, less than 8 ng/ml, less than 6 ng/ml, less than 5 ng/ml, less than 3 ng/ml or even less than 1 ng/ml). In some cases, the cells are expanded in the presence of exogenously added IL-15 (e.g., at least 10 ng/ml) and minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml). minimal or no exogenously added IL-7 (e.g., less than 10 ng/ml, less than 8 ng/ml, less than 6 ng/ml, less than 5 ng/ml, less than 3 ng/ml or even less than 1 ng/ml) and minimal or no exogenously added IL-21 (e.g., less than 10 ng/ml, less than 8 ng/ml, less than 6 ng/ml, less than 5 ng/ml, less than 3 ng/ml or even less than 1 ng/ml). In some cases, the only exogenously added interleukin is IL-15 (preferably human IL-15). In some cases, all exogenously added interleukins other than IL-15 (e.g., IL-7, IL-21, IL-4 and IL-9) are present at less than 10 ng/ml (less than 8 ng/ml, less than 6 ng/ml, less than 5 ng/ml, 3 ng/ml or even less than 1 ng/ml) and exogenously added IL-2 is present at less than 50 U/ml (less than 40 U/ml, less than 30 U/ml, less than 20 U/ml, less than 10 U/ml, less than 5 U/ml or even less than 1 U/ml). Exogenously added interleukins are those that are added to the culture media as opposed to being generated by the cells themselves.

The T cell populations that can be expanded using the manufacturing methods described herein can include: naïve T cells (T_{N}), memory stem cells (T_{SCM}), central memory T cells (T_{CM}) and combinations thereof in addition to other cells such as effector T cells (T_{E}) or effector memory T cells (T_{EM}). FIG. 1 schematically depicts these cells type and certain of the cell surface markers expressed by each. T cell populations that are primarily naïve T cells (T_{N}), memory stem cells (T_{SCM}), and central memory T cells (T_{CM}) with few T_{E} and T_{EM} cells can be described as T_{CM/SCM/N} cells or T_{CM/SCM/N} cell populations. These cell populations can be derived from peripheral blood mononuclear cells (PBMC) by both: 1) depleting unwanted cell populations such as CD14 expressing myeloid cells and CD25 expressing cells; and 2) enriching for CD62L expressing memory and naïve T cells. Thus, the resulting population of cells includes T naïve (T_{N}) and stem memory cells (T_{SCM}) expressing CD45RA and CD62L. It also includes the population of central memory T cells (T_{CM}) that express CD45RO and CD62L. T_{CM/SCM/N} cell populations differ from previously described T_{CM} cell populations in that their preparation does not entail depletion of CD45RA+ T cells. T_{CM/SCM/N} cell populations, upon preparation, are relatively free of effector memory cells (T_{EM}) and effector cells (T_{E}). In addition, such T cell populations have a relatively high proportion or CD45RA+CD45RO− T cells.

In certain embodiments of the manufacturing method described herein, a population of T cells, e.g., a T_{CM/SCM/N} cell population, a T_{CM} cell population, a T_{N} or unselected PBMC, is stimulated and transduced with a vector expressing a desired T cell receptor, e.g., a CAR. After transduction, the cells are expanded by culturing in a medium comprising exogenously added IL-15 at greater than or equal to 5 ng/ml or 10 ng/ml and exogenously added IL-2 at less than or equal to 50, 40, 30, 20 or 10 U/ml (“High IL-15/Low IL-2 culture conditions”). In some cases, exogenously added IL-7 and or exogenously added IL-21 are each present at less than 10 ng/ml (less than 5 ng/ml, 3 ng/ml or 1 ng/ml or there is no exogenously added IL-2, IL-7 or IL-21). As the T cells are expanded over a period of days, differentiation will occur giving rise to, for example, additional T_{E} cells and additional T_{EM} cells. Thus, where the starting T cell population is a T_{CM/SCM/N} cell population, culturing will, over time lead to an increase in the proportion of CD45RA+CD45RO+ T cells and the proportion of CD45RA−CD45RO+ T cells. However, compared to certain conventional culture conditions with relatively low exogenously added IL-15 and relatively high exogenously added IL-2, the High IL-15/Low IL-2 culture conditions described herein result in a higher proportion of desirable CD45RA+CD45RO− T cells. In addition, as demonstrated herein, T cell populations expressing a CAR expanded under the High IL-15/Low IL-2 culture conditions express a lower level of exhaustion markers such as 2B4 and Lag3.

In some cases, the cells are cultured in High IL-15/Low IL-2 (or High IL-15/Low IL-2, IL-7, IL-21 conditions) during activation (i.e., when the cells are being activated, for example by CD28/CD3 beads for transduction). In some cases, there are no exogenously added interleukins present during activation.

The manufacturing methods described herein can be used to expand T cell populations for a variety of therapeutic purposes. For example, the methods can be used to expand tumor infiltrating lymphocytes (TIL) isolated from a patient.

The manufacturing methods described herein can be used to expand a T cell population that is subsequently transfected with an RNA (e.g., an mRNA) encoding a T cell receptor (Krug et al. 2014 Cancer Immunology and Immunotherapy 63:999)

Patient-specific, autologous and allogeneic T cells (e.g., autologous or allogenic T_{CM/SCM/N} cells) can be engineered to express a chimeric antigen receptor (CAR) or T cell receptor (TCR) and the engineered cells can be expanded under High IL-15/Low IL-2 culture conditions or High IL-15/Low IL-2, IL-7, IL-21 conditions.

Described herein is a method for expanding T cells in culture medium that includes exogenously added IL-15 and little or no exogenously added IL-2 (and, optionally, little or no exogenously added IL-7 or IL-21). Also described is a method for activating a population of T cells that are cultured in culture media that includes exogenously added IL-15 and little or no exogenously added IL-2 (and, optionally, little or no exogenously added IL-7 or IL-21). Also described is a method for introducing a vector, e.g., a lentiviral or retroviral vector, expressing a T cell receptor (e.g., a CAR) into a population of T cells that have been activated and the expanding the cells in culture media that includes exogenously added IL-15 and little or no exogenously added IL-2 (and, optionally, little or no exogenously added IL-7 or IL-21). The introduction of the vector can take place in culture media that includes exogenously added IL-15 and little or no exogenously added IL-2 (and, optionally, little or no exogenously added IL-7 or IL-21) and includes components to cause T cell activation (e.g., CD3/CD28 beads). Alternatively, activation can take place in the absence of exogenously added interleukins.

Described herein is a method for preparing a population of human cells comprising T cells (i.e., CD3+ cells) optionally harboring a recombinant nucleic acid molecule encoding a T cell receptor, comprising: (a) providing a sample of human cells comprising T cells, wherein the T cells comprise: central memory T cells; memory stem T cells, and naïve T cells, wherein greater than 40% (greater than 45%, 50%, 55%, 60%, 65% or 70%) of the T cells are CD45RA+ and greater than 70% (greater than 75%, 80%, 85% or 90%) of the T cells are CD62L+; (b) activating the population of human cells comprising T cells; and (c) transducing or transfecting cells in the population of human cells comprising T cells with a recombinant nucleic acid molecule to provide a population of human cells comprising T cells harboring a recombinant nucleic acid molecule, wherein the method does not comprise a step of depleting cells expressing CD45RA, and then expanding the cells in culture media that includes exogenously added IL-15 and little or no exogenously added IL-2 (and, optionally, little or no exogenously added IL-7 or IL-21). In various embodiments: the recombinant nucleic acid molecule is a viral vector (e.g., a lentiviral vector or a retroviralvector encoding a T cell receptor such as a CAR); the method further comprises culturing the population of human cells comprising T cells harboring a recombinant nucleic acid molecule; the culturing step comprises the addition of exogenous IL-2 and exogenous IL-15 (and, optionally, little or no exogenously added IL-7 or IL-21); and the activating step comprises exposing the cells to an anti-CD3 antibody and an anti-CD28 antibody; and at least 80% (greater than 85%, 90%, 95%, or 98%) of the cells in the isolated population of cells comprising T cells are T cells. In some embodiments, step (c) is omitted and the cells are treated subsequent to expansion to introduce an RNA molecule encoding a T cell receptor such as a CAR. The RNA can be introduced into the expanded cells by electroporation or another suitable method and the transfected cells will transiently express the T cell receptor.

Described herein is method for preparing a population of human cells comprising T cells (i.e., cells that express CD3 or CD3+ cells), wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, comprising: (a) providing an isolated population of human cells comprising T cells; (b) treating the isolated population of human cells comprising T cells to deplete cells expressing CD25 and cells expressing CD14 to prepare a depleted cell population; and (c) treating the depleted cell population to enrich for cells expressing CD62L, thereby preparing a population of human cells comprising T cells, wherein the T cells comprise central memory T cells; memory stem T cells, and naïve T cells, wherein greater than 40% of the cells are CD45RA+ (greater than 45%, 50%, 55%, 60%, 65% or 70%) and greater than 70% are CD62L+ (greater than 75%, 80%, 85% or 90%), wherein the method does not comprise a step of depleting cells expressing CD45RA, and then expanding the cells in culture media that includes exogenously added IL-15 and little or no exogenously added IL-2.

The population of T cells expanded in culture media that includes exogenously added IL-15 and little or no exogenously added IL-2 (and, optionally, little or no exogenously added IL-7 or IL-21) can be a population of human cells comprising T cells (i.e., cells that express CD3 or CD3+ cells), wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, wherein the population is prepared by a method comprising: providing an isolated population of human cells comprising T cells (e.g. PBMC from a donor); treating the isolated population of human cells comprising T cells to deplete cells expressing CD25 and deplete cells expressing CD14 to prepare a depleted cell population; and treating the depleted cell population to enrich for cells expressing CD62L, thereby preparing a population of human cells comprising T cells, wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, wherein the method does not comprise a step of depleting cells expressing CD45RA. In various embodiments: less than 15% (less than 12%, 10%, 8%, 6%) of the T cells in the population of human cells are CD14+ and less than 5% (less than 4%, 3% or 2%) of the T cells are CD25+; at least 40% (greater than 45%, 50%, 55%, 60%, 65% or 70%) of the T cells are CD4+ and CD62L+ or CD8+ and CD62L+; at least 10% (greater than 15%, 20%, 25%, 30%, 35%, or 40%) of the T cells are CD8+ and CD62L+; less than 60% (less than 55%, 50%, 45%, 40%, 35%, 30%, 24%, 20% or 15%) of the T cells are CD45RO+. The population of T cells can be primarily CD4+ cells (greater than 60, 70, 80 or 90% CD4+ cells) or primarily CD8+ cells (greater than 60, 70, 80 or 90% CD8+ cells).

Also described herein is a method of treating cancer, autoimmunity or infection comprising administering to a patient in need thereof a pharmaceutical composition comprising a human cell population manufactured under High IL-15/Low IL-2 culture conditions or High IL-15/Low IL-2, IL-7, IL-21 conditions described herein.

In some cases, the cells are autologous to the patient being treated and in some cases they are allogenic to the patient being treated.

### BRIEF DESCRIPTION OF THE DRAWINGS

FIG. 1 depicts certain marker expression data for various T cell subsets.

FIG. 2 schematically depicts the generation and culturing of a T cell populations used in the studies described in FIGS. 3-6.

FIG. 3 depicts the results of studies showing that CAR T cells expanded in the presence of IL-15 and in the absence of exogenously added IL-2 have improved in vivo antitumor activity. (A) Schematic representation of the experimental design to assess the antitumor effects of CD19 targeted CAR-T cells expanded with various cytokine combination. The CD19 CAR has been previously reported (Wang et al. 2015 Clinical Cancer Research 21:2993; Jonnalagadda et al. 2014 Molecular Therapy 23:757) and is comprised of the FMC63 scFv, a modified IgG4-Fc linker mutated, CD28 transmembrane domain and CD28-CD3ζ endodomains. The CAR cassette also includes a T2A ribosomal skip sequence followed by a truncated EGFRt for cell to detect transduced cells. (B) Bioluminescence imaging of tumor progression in mice engrafted with Raji tumor cells and treated with CAR T cells. (C) Kaplan Meier survival curve of mice after infusion of CAR T cells.

FIG. 4 depicts the results of studies showing that CAR T cells expanded in IL-15 in a long-term culture sustain their antitumor activity. CAR T cells were expanded in either IL-2 or IL-15 cytokine. At various time-points, cells were collected and assessed for their antitumor activity against Raji tumors in vivo. (A) Bioluminescence imaging (BLI) of tumor progression in mice engrafted with Raji tumor cells and treated with CAR T cells. (B) Photon flux of tumor cells with and without treatment acquired via BLI at various time-points (n=6). (C) Kaplan Meier survival curve of mice after infusion of CAR T cells.

FIG. 5 depicts the results of a study showing that IL-15 preserves less-differentiated memory phenotype of CAR T cells during ex vivo expansion. CAR T cells were expanded in either IL-2 or IL-15 cytokine. At various time-points, cells were collected and assessed for changes in memory phenotype. T cells were harvested on days 14 and 32 and flow cytometry analysis of their phenotype was conducted. (A) Pie chart shows reduction in frequency of CD45RA+CD62L+ cells cultured in IL-2 over time. (B) Flow cytometry analysis shows sustained CD27 expression in T cells cultured in IL-15

FIG. 6 depicts the results of a study showing that IL15 prevents expression of T cell exhaustion markers over long-term ex vivo culture. T cells cultured in either IL2 or IL15 cytokines were analyzed for exhaustion phenotypes on days 14, 23 and 32. Flow cytometry analysis shows over time increased expression of Lag3 (Top) and 2B4 (bottom) in T cells cultured in IL2. Graphs are summary data obtained from two different donors.

### DETAILED DESCRIPTION

The T cell compartment includes T cell subsets that are at different stages of differentiation. These subsets arise from differentiation of Naïve T cells (T_{N}), which are CD45RA+, CD62L+, CD28+, and CD95−. Among the stem cell-like subsets are Memory Stem Cells (T_{SCM}), which are CD45RA+, CD62L+, CD28+, and CD95+. These cells differentiate into Central Memory Cells (T_{CM}), which are CD45RO+, CD62L+, CD28+, and CD95+. T_{CM} differentiate in Effector Memory Cells (T_{EM}), which are CD45RO+, CD62L−, CD28+/−, and CD95+. The T_{EM} differentiate to Effector T cells (T_{E}) which are CD45RO+, CD62L+, CD28+, and CD95+.

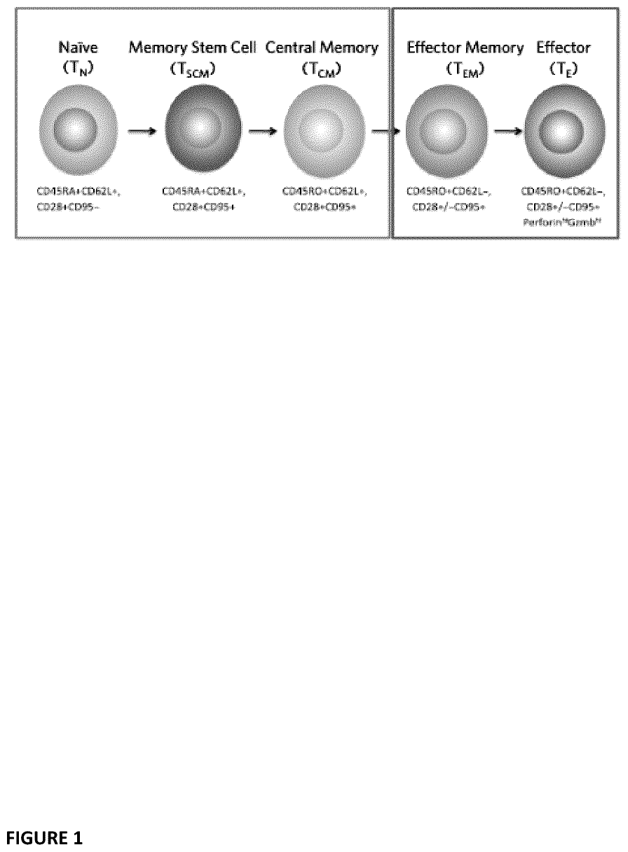

Memory Stem T Cells (T_{SCM}) are present at a low level in the T cell compartment, but appear to have significant self-renewal and proliferative potential. While they resemble naïve T cells (T_{N}) in that they express CD45RA+and CD62L+, they can be distinguished from T_{N} by their expression of CD95 (FIG. 1). T_{SCM} can be generated from T_{N} by stimulation with CD3/CD28 beads in the presence of IL-7 and IL-15. They also can be expanded in the presence of Wnt/β-catenin pathway activation (Cieri et al. 2013 Blood 121:573; Gattinoni et al. 2009 Nature Medicine 15:808).

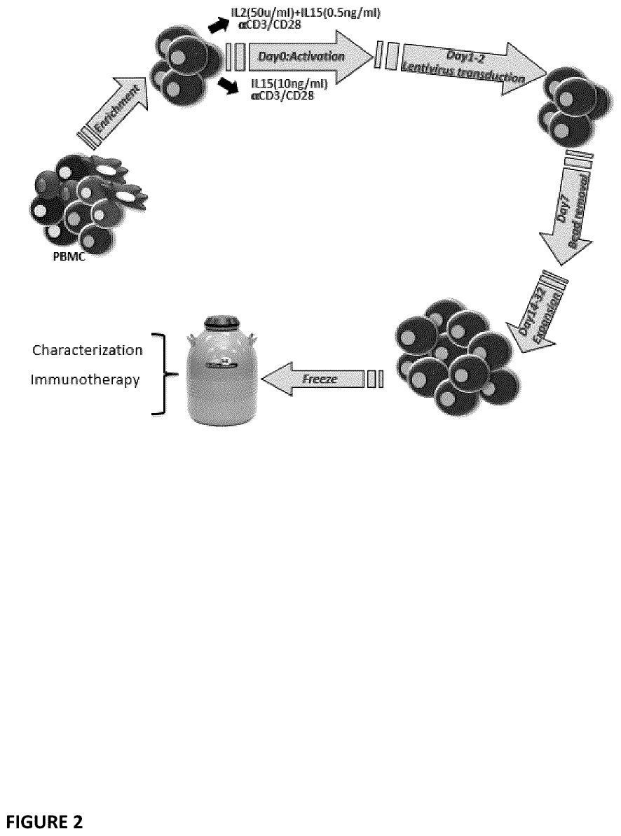

Central Memory T Cells (T_{CM}), which are more abundant in PBMC than are T_{SCM}, are a well-defined memory T cell subset with high self-renewal and proliferative potential. There is evidence that T_{CM} persist following adoptive transfer better than Effector T cells (T_{E}) (Berger et al. 2008 Journal of Cellular Immunology 118:4817; Wang et al 2011 Blood 117:1888). T_{CM} can be enriched from PBMC for T cell therapy manufacturing based on their CD45RA− CD45RO+ CD62L+ phenotype (FIG. 2) (Wang et al. 2012 J Immunotherapy 5:689). There is some evidence that T_{CM} behave as adult stem cells. Studies in mice demonstrated that: single cell transfer of T_{CM} over three generations demonstrated that T_{CM} can provide full immune reconstitution; that T_{CM} expand to produce more T_{CM}; and that T_{CM} differentiate to T_{EM}/T_{E} (Graef et al. 2014 Immunity 41:116; Gattioni et al. 2014 Immunity 41:7).

The various T cell populations described can be genetically engineered to express, for example, a CAR or a T cell receptor. A CAR is a recombinant biomolecule that contains an extracellular recognition domain, a transmembrane region, and one or more intracellular signaling domain. The term “antigen,” therefore, is not limited to molecules that bind antibodies, but to any molecule that can bind specifically to any receptor. “Antigen” thus refers to the recognition domain of the CAR. The extracellular recognition domain (also referred to as the extracellular domain or simply by the recognition element which it contains) comprises a recognition element that specifically binds to a molecule present on the cell surface of a target cell. The transmembrane region anchors the CAR in the membrane. The intracellular signaling domain comprises the signaling domain from the zeta chain of the human CD3 complex and optionally comprises one or more co-stimulatory signaling domains. CARs can both to bind antigen and transduce T cell activation, independent of MHC restriction. Thus, CARs are “universal” immunoreceptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

The CAR can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region can be inserted into an expression vector and used to transform a suitable expression host cell line, preferably a T lymphocyte cell line, and most preferably an autologous T lymphocyte cell line. Alternatively, the coding region can be transiently expressed by an RNA that is introduced into the T cells after expansion using the methods described herein.

Various CAR suitable for expression by T_{CM/SCM/N} cells include, for example, those described in: WO 2016/044811; WO 2104/144622; WO 2002/077029; and WO/US2014/0288961.

### Example 1: Preparation of T_{CM/SCM/N} Cells

A variety of methods can be used to produce a population of human T_{CM/SCM/N} cells. For example, a population of T_{CM/SCM/N} cells can be prepared from a mixed population T lymphocytes. The population of T lymphocytes can be allogenic to or autologous to the subject ultimately treated using the cells and can be obtained from a subject by leukopheresis or blood draw.

The following method is an example of one that can be used to obtain a population of T_{CM/SCM/N} cells from T lymphocytes obtained by leukapheresis or other means. Peripheral blood is collected by leukapheresis or peripheral blood draw. Day 1 of a typical manufacturing cycle is the day the ficoll procedure takes place. The subject's leukapheresis product is diluted with EDTA/PBS and the product is centrifuged at 1200 RPM for 10 minutes at room temperature with maximum brake. After centrifugation, the platelet-rich supernatant is removed and the cell pellet is gently vortexed. EDTA/PBS is used to re-suspend the vortexed cell pellets in each conical tube. Each tube is then underlayed with ficoll and centrifuged at 2000 RPM for 20 minutes with no brake at room temperature. Following centrifugation, the PBMC layer from each tube is transferred into another conical tube. The cells are centrifuged at 1800 RPM for 15 minutes with maximum brake at 4° C.

After centrifugation, the cell-free supernatant is discarded and the cell pellet is gently vortexed. The cells are washed twice using EDTA/PBS each time, and a third time using PBS. Cells are centrifuged each time at 1200 RPM for 10 minutes with maximum brake at 4° C. After the final PBS wash, the vortexed cell pellet is resuspended in complete X-VIVO 15 media (X-VIVO™ media with 10% FBS) and transferred to a transfer bag. The bag with washed PBMC is kept overnight on a rotator at room temperature on the bench top for immunomagnetic selection the next day.

Next, selection procedures are used to both to deplete the cell population of cells expressing certain markers and to enrich the cell population for cells expressing certain other markers. These selection steps preferably occur on day two of the manufacturing cycle. The cell population is substantially depleted for cells expressing CD25 and CD14. Importantly, the cell population is not substantially depleted for cells expressing CD45RA. Briefly, cells resuspended in labeling buffer (LB; EDTA/PBS with 0.5% HSA), and incubated with anti-CD14 and anti-CD25 Miltenyi antibodies for CliniMACS® depletion, and the composition is gently mixed and then incubated for 30 minutes on a rotator at room temperature on the bench top.

The depletion step is performed on a CliniMACS® device using a depletion tubing set. The recovered cells following the depletion step are transferred into tubes and centrifuged at 1400 RPM for 15 minutes with maximum brake at 4° C.

The cell-free supernatant is removed and the cell pellet is gently vortexed and resuspended. To enrich for cells expressing CD62L, the cell suspension is treated with anti-CD62L-biotin (made at the City of Hope Center for Biomedicine and Genetics), gently mixed and incubated for 30 minutes on a rotator at room temperature on the bench top.

Following the incubation period, LB is added to the tube and cells are centrifuged at 1400 RPM for 15 minutes at maximum brake at 4° C. The cell-free supernatant is removed and the cell pellet is gently vortexed. LB is added to resuspend the cell pellet in the tube and the resuspended cells are transferred to a new transfer bag. Anti-biotin (Miltenyi Biotec) reagent is added and the mixture is gently mixed and incubated for 30 minutes on a rotator at room temperature on the bench top

The CD62L enrichment step is performed on a CliniMACS® device using a tubing set. The product of this enrichment can be frozen for storage and later thawed and activated

To provide an intermediate holding step in the manufacturing, the option exists to freeze cells following the selection process. The cells are pelleted by centrifugation at 1400 RPM for 15 minutes with max break at 4° C. The cells are resuspended in Cryostor® and aliquoted into cryovials. The vials are transferred to a controlled cooling device that can cool at about 1° C./minute (e.g., a Nalgene® Mr. Frosty; Sigma-Aldrich) the cooling device is immediately transferred to a −80° C. freezer. After three days in the −80° C. freezer, the cells are transferred into a GMP LN2 freezer for storage.

We have found that cryopreserved cells exhibit good recovery and viability, maintain the appropriate cell surface phenotype when thawed up to 8.5 months after cryopreservation, and can be successfully transduced and expanded in vitro upon thawing.

Alternatively, freshly enriched T_{CM/SCM/N} cells can be activated, transduced and expanded as described below.

### Example 2: Activation, Lentiviral Transfection and Culturing in the Presence of Certain Cytokines

Human T cells, either bulk PBMC or enriched T cell subsets, are stimulated as for example with GMP Dynabeads® Human T expander CD3/CD28 (Invitrogen) at a 1:3 ratio (T cell:bead). On day 0 to 3 of cell stimulation, T cells are transduced, for example with a CAR-expressing lentivirus, in X Vivo15 containing 10% fetal calf serum (FCS) with 5 μg/mL protamine sulfate (APP Pharmaceutical), and with exogenously added cytokines (i.e., final concentration 10 ng/mL rhIL-15). The next day following lentivirus transduction, media is exchanged or cultures diluted 1:2 to in X Vivo 15 containing 10% FCS and cytokines. Cultures are then maintained at 37° C., 5% CO₂ with addition of X-Vivo15 10% FCS as required to keep cell density between 3×10⁵ and 2×10⁶ viable cells/mL, with cytokine supplementation (i.e, final concentration of 10 ng/mL rhIL-15) every Monday, Wednesday and Friday of culture. On day 7 to 10 following T cell stimulation, the CD3/CD28 Dynabeads are removed from cultures using the DynaMag-50 magnet (Invitrogen). Cultures are propagated until day 8 to 32 days and then cryopreserved. Over the duration of the culture, cells are supplemented with a combination of cytokines [IL2 (50 U/mL)+IL15 (0.5 ng/mL), IL7 (10 ng/ml)+IL15 (10 ng/mL) or IL7 (10 ng/mL)+IL15 (10 ng/mL)+IL21 (10 ng/ml), or IL-15 only (10 ng/ml). Two thirds of the culture media is removed and fresh media consisting of above cytokine combination is added at a 0.6×10⁶ cells/mL concentration. Exogenous cytokine addition is optional during the CD3/CD28 bead stimulation phase, however, it is essential during the expansion phase following removal of the beads. The amount of cytokine added to reach a desired level of exogenously added cytokine is based in the assumption that any media not replaced when fresh media is added is essentially free of any previously exogenously added cytokine.

### Example 3: CAR T Cells Expanded in the Presence of IL-15 and in the Absence of Exogenously added IL-2 have Improved In Vivo Antitumor Activity

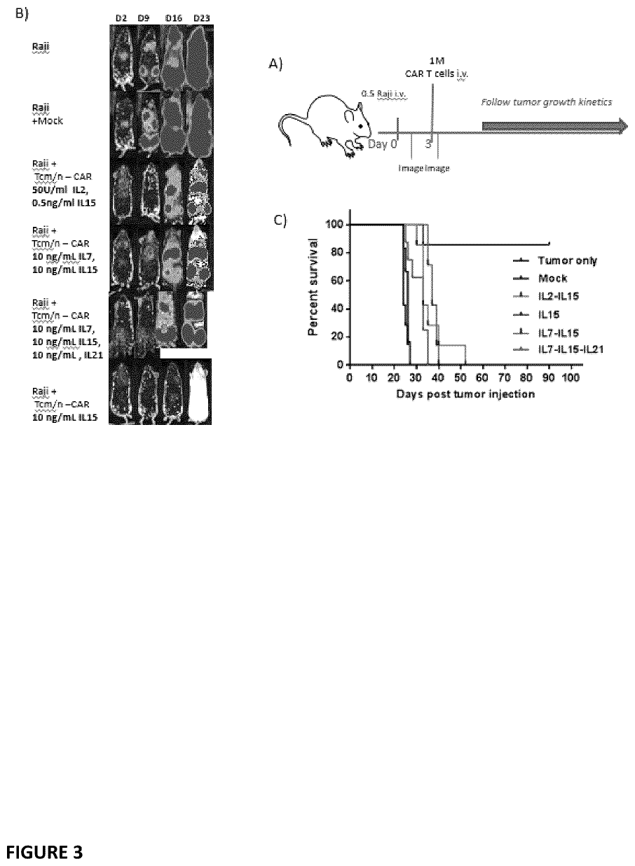

T_{CM/SCM/N} cells prepared and transduced as described above to express a CAR targeted to CD19 were expanded in the presence of 50 U/ml of IL-2 and 0.5 ng/ml of IL-15; 10 ng/ml of each of IL-7 and IL-15; 10 ng/ml of each of IL-7, IL-15 and IL-21 or 10 ng/ml of IL-15 only. The cells were injected into mice engrafted with Raji tumor cells. The experimental design is shown schematically in FIG. 3(A). Bioluminescence imaging of tumor progression in mice engrafted with Raji tumor cells and treated with CAR T cells is shown in FIG. 3(B) and Kaplan Meier survival curve of mice after infusion of CAR T cells is shown in FIG. 3(C).

As can be seen, in all conditions except IL-15 only, there was less than 50% survival by day 40. Importantly, excluding IL-2 and excluding IL-7 when IL-15 was present, improved anti-tumor activity.

### Example 4: CAR T Cells Expanded Long Term in the Presence of IL-15 and in the Absence of Exogenously added IL-2 Sustain In Vivo Antitumor Activity

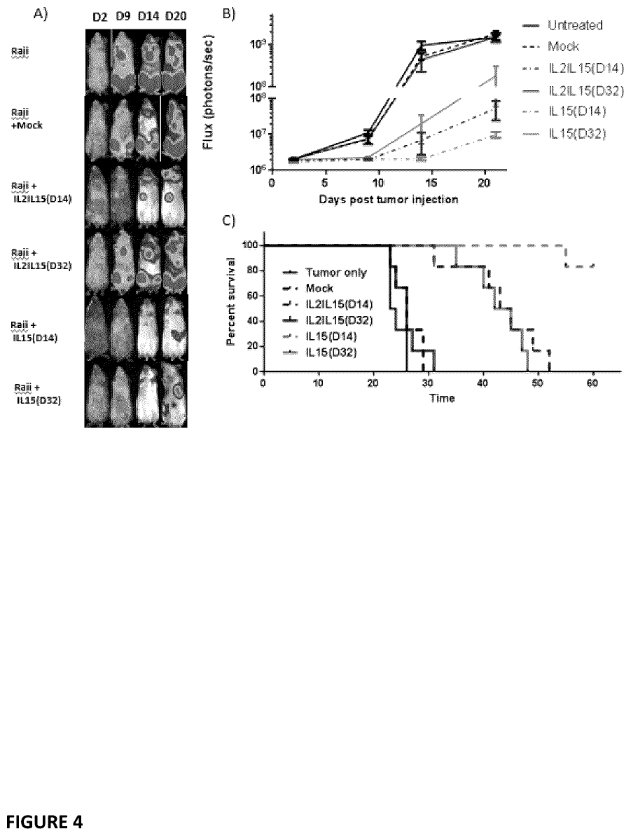

As shown in FIG. 4(A)-(C), CAR T cells expanded in IL-15 in a long-term culture sustain their antitumor activity. CAR T cells were expanded in either IL-2 (50 U/ml) with low IL-15 (0.5 ng/ml) or IL-15 only (10 ng/ml). At various time-points, cells were collected and assessed for their antitumor activity against Raji tumors in vivo. FIG. 4(A) depicts bioluminescence imaging of tumor progression in mice engrafted with Raji tumor cells and treated with CAR T cells. FIG. 4(B) present photon flux of tumor cells with and without treatment acquired via bioluminescence imaging at various time-points (n=6) and FIG. 4(C) presents Kaplan Meier survival curve of mice after infusion of CAR T cells.

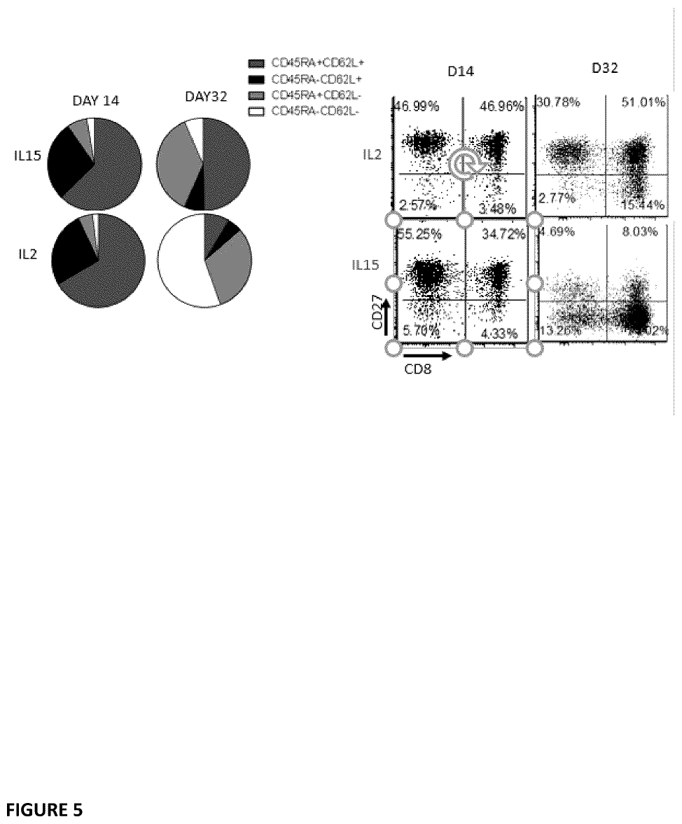

FIG. 5 depicts the results of a study showing that IL-15 preserves less-differentiated memory phenotype of CAR T cells during ex vivo expansion. CAR T cells were expanded in either IL-2 at 50 U/ml with low IL-15 (0.5 ng/ml) or IL-15 at 10 ng/ml. At various time-points, cells were collected and assessed for changes in memory phenotype. T cells were harvested on days 14 and 32 and flow cytometry analysis of their phenotype was conducted. (A) Pie chart shows reduction in frequency of CD45RA+CD62L+ cells cultured in IL-2 over time. (B) Flow cytometry analysis shows sustained CD27 expression in T cells cultured in IL-15.

As can be seen when cells were expanded in the presence of IL-2 at 50 U/ml with low IL-15 (0.5 ng/ml) for 14 days, 50% survival was between 45 and 50 days, but this decreased to between 25 and 30 days when the cells were expanded for 32 days. In contrast, for cells expanded in IL-15 only at 10 ng/ml, 50% survival was between 45 and 50 days even when the cells had been expanded for 14 days and was far longer when the cells were expanded for 14 days.

### Example 5: Expansion in the Presence of IL-15 Preserves Less-Differentiated Memory Phenotype of CAR T Cells Compared to Expansion in the Presence of IL-2

CAR T cells were expanded in either IL-2 (50 U/ml) with low IL-15 (0.5 ng/ml) or IL-15 (10 ng/ml). At various time-points, cells were collected and assessed for changes in memory phenotype. T cells were harvested on days 14 and 32 and flow cytometry analysis of their phenotype was conducted. FIG. 5(A) is shows reduction in frequency of CD45RA+CD62L+ cells cultured in IL-2 with low IL-15 over time. Cells culture in the presence of IL-15 only showed a higher proportion of CD45RA+CD62L+ cells. Flow cytometry analysis showed sustained CD27 expression in T cells cultured in IL-15 (FIG. 5(B)). Together this data indicates that IL-15 preserves the memory stem cell phenotype. Less differentiated T cell product has longer persistence and potentially enhanced self-renewal.

### Example 6: Expansion in the Presence of IL-15 Reduces Expression of Exhaustion Markers During Long Term Ex Vivo Culture Compared to Expansion in the Presence of IL-2

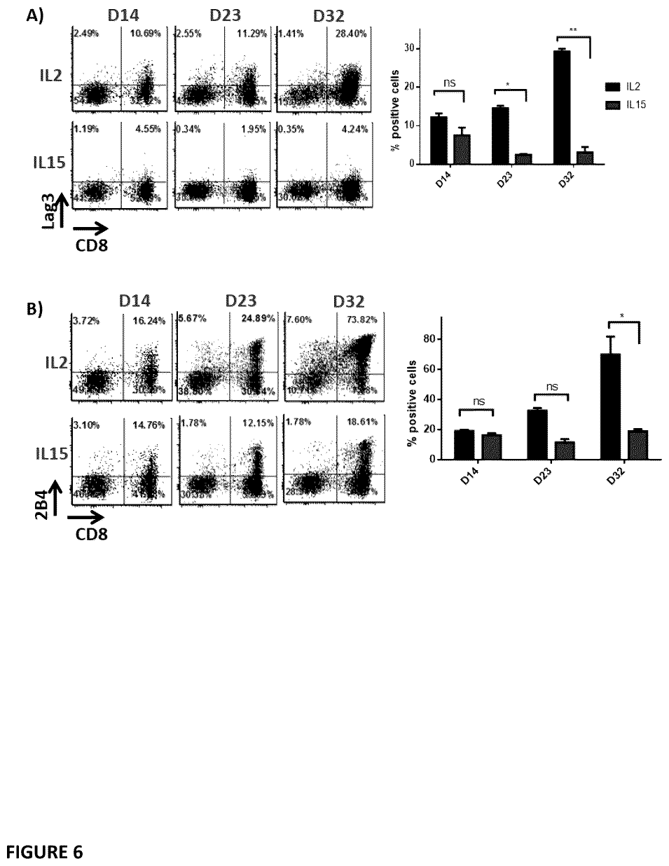

T cells cultured in either 50 U/ml of IL-2 with low IL-15 (0.5 ng/ml) or 10 ng/ml of IL-15 were analyzed for exhaustion phenotypes on days 14, 23 and 32. Flow cytometry analysis shows over time increased expression of Lag3 (FIG. 6(A)) and 2B4 (FIG. 6(B)) in T cells cultured in IL-2. Exhaustion is a major defect in limiting T cell function. T cells with exhausted phenotype have impaired proliferation, persistence, and antitumor efficacy after adoptive transfer.

1-39. (canceled)

40. A method for manufacturing a T cell population, comprising:
(a) obtaining a sample of PBMC from a human patient;
(b) treating the sample of PBMC to: deplete cells expressing CD14, deplete cells expressing CD25, and enrich for cells expressing CD62L to create a population of enriched T cells; and
(c) culturing the population of enriched T cells in culture medium comprising exogenously added IL-15 at a concentration of at least 5 ng/ml and in the absence of both exogenously added IL-2 and exogenously added IL-7.
wherein the manufacturing method does not comprise depleting cells expressing CD45RA.

41. The method of claim 40 further comprising introducing a nucleic acid molecule into at least a portion of the cells of the enriched T cell population.

42. The method of claim 41, wherein the nucleic acid molecule is introduced to the population of enriched T cells before step (c).

43. The method of claim 41, wherein the nucleic acid molecule encodes a chimeric antigen receptor (CAR), thereby creating CAR T cells.

44. The method of claim 43, wherein greater than 40% of the CAR T cells express CD45RA and greater than 70% of the CAR T cells express CD62L.

45. The method of claim 40, wherein exogenously added IL-15 is present at a concentration of at least 10 ng/ml.

46. The method of claim 40, wherein the concentration of exogenously added IL-15 in the culture medium is no more than 100 ng/ml, 90 ng/ml, 80 ng/ml, 70 ng/ml. 60 ng/ml, 50 ng/ml, 40 ng/ml, 30 ng/ml, 20 ng/ml, or 15 ng/ml.

47. The method of claim 40, wherein the culture medium comprises exogenously added IL-21 at concentration of less than 1 ng/ml.

48. The method of claim 40, wherein the culture medium comprises no exogenously added IL-21.

49. The method of claim 40, the population of enriched T cells is cultured in the culture medium for at least five days and less than 40 days.

50. The method of claim 40, wherein the population of enriched T cells is cultured for a period of time sufficient to expand the population less than 100-fold.

51. The method of claim 40, further comprising culturing the population of enriched T cells in the presence of antibodies targeted to human CD3 and antibodies targeted to human CD28 prior to step (c).

52. The method of claim 40, wherein at least 30% of the T cells in the enriched population of T cells express CD4 and at least at least 10% of the T cells in the enriched population of T cells express CD8.

53. The method of claim 40, wherein at least 70% of the cells in the enriched T cell population are CD4+ T cells.

54. The method of claim 40, wherein at least 70% of the cells in the enriched T cell population are CD8+ T cells.

55. The method of claim 40, wherein at least 25% of the T cells in the enriched population of T cells express CD45RA.

56. The method of claim 40, wherein at least 50% of the T cells in the enriched population of T cells express CD62L.

57. The method of claim 40, wherein no more than 50% of the T cells in the enriched population of T cells are CD62L−.

58. The method of claim 40, wherein greater than 40% of the enriched T cells express CD45RA and greater than 70% of the enriched T cells express CD62L.
